(19) United States
(12) Patent Application Publication
(10) Pub. No.: US 2014/0294736 A1
(43) Pub. Date: Oct. 2, 2014

(54) TOOTHPASTE

(71) Applicant: Givaudan S.A., Vernier (CH)

(72) Inventors: Mary Amanda Mckee, Cincinnati, OH (US); Theodore T. Pearl, Harrison, OH (US)

(73) Assignee: Givaudan S.A., Vernier (CH)

(21) Appl. No.: 14/356,827

(22) PCT Filed: Nov. 12, 2012
(86) PCT No.: PCT/EP2012/072371

§ 371 Date: May 7, 2014

Related U.S. Application Data
(60) Provisional application No. 61/558,781, filed on Nov. 11, 2011.

(51) Int. Cl. A61K 8/02; A61Q 11/00

(52) U.S. Cl. CPC A61K 8/0245; A61Q 11/00
USPC 424/10.4

(57) ABSTRACT

A dental brushing composition including a brushing composition base additionally containing a plurality of microparticles, the particles comprising a core, a coating essentially completely surrounding the core, and two different flavors, a general flavor and an indicator flavor, the general flavor being in the coating and the indicator flavor in the core, the coating being essentially completely soluble under brushing conditions in a desired brushing time. The composition signals when a desired brushing time has been achieved by a noticeable change in flavor.

This disclosure relates to dental brushing compositions and to methods of timing the use of these products.

Regular brushing of teeth with a brushing composition, such as a toothpaste or tooth gel, is essential to good dental hygiene and prevention of decay. The optimum brushing time for teeth is not capable of precise definition, but dentists are generally of the opinion that the appropriate brushing time is about 2 minutes. While exceeding this time does not make dental hygiene any better, a shorter time can be detrimental, yet this is what increasing numbers of people do, especially those with busy schedules. A dental brushing composition that gives some indication as to when an optimal brushing has been achieved would therefore be very useful.

Such compositions are already known in the art. The indication may be provided by, for example, a change of color (for example, U.S. Pat. No. 6,419,942), or a change of flavor (for example, U.S. Pat. No. 3,957,964 and French Demande 2 643 261). Both of the named flavor-change documents utilise microencapsulated flavors, which are adapted to release the flavor at a desired time. In the case of U.S. Pat. No. 3,957,964, two flavors are released at different times, by utilising two different populations of capsules, the natures of whose walls are such that the flavors are released at different times. This has the advantage of preserving the flavors during storage and releasing them only when necessary.

It has now been found that it is possible to make a dental brushing composition in which the appropriate brushing time is indicated by a flavor change, but realised in a more practical way. There is therefore provided a dental brushing composition comprising a brushing composition base additionally containing a plurality of microparticles, the microparticles comprising a core, a coating essentially completely surrounding the core, and two different flavors, a general flavor and an indicator flavor, the general flavor being in the coating and the indicator flavor in the core, the coating being essentially completely soluble under brushing conditions in a desired brushing time.

There is additionally provided a method of indicating the attainment of a desired brushing time with a brushing dental composition, comprising the release into a mouth of two different encapsulated flavors, a general flavor and an indicator flavor, the indicator flavor indicating the attainment of the brushing time, characterised in that both flavors are incorporated in the same microparticles, which microparticles each comprise a core and a coating essentially completely surrounding the core, the core comprising the indicator flavor and the coating the general flavor, the coating being essentially completely soluble under brushing conditions in a desired brushing time.

There is additionally provided a method comprising providing a brushing dental composition capable of indicating the attainment of a desired brushing time therewith, said indicating comprising releasing into a mouth two different encapsulated flavors, wherein the two different flavors comprise a general flavor and an indicator flavor, the indicator flavor indicating the attainment of the brushing time, characterised in that both flavors are incorporated in a single population of microparticles, the microparticles comprising a core and a coating essentially completely surrounding the core, the core comprising the indicator flavor and the coating comprising the general flavor, the coating being essentially completely soluble under brushing conditions in the desired brushing time.

The compositions hereinabove described have the advantage that only one population of microparticles need be made. In addition, the microparticles are more robust than conventional microcapsules, and there is therefore less loss of flavor during storage and rough handling.

Apart from the fact that the general and indicator flavors must be different, that is, must be perceived thus by the consumer utilising the compositions, the nature of the flavors is not narrowly critical, and they can be chosen from among the wide variety of such flavors known to and used by the art. Dental products generally utilise mint flavors, which give a desirable clean, fresh taste, and so two different mint flavors may be used, for example spearmint, peppermint and wintergreen. However, mint is not essential, and other flavors, such as fruit flavors, may be used as one or both of the general and indicator flavors.

Non-limiting examples of suitable flavors include the mint flavors previously mentioned, citrus (orange lemon, lime, grapefruit, etc.) berries (strawberry, blueberry, blackberry, etc.), other fruits (bananas, apples, melon, etc.), and fruit flavor mixtures (tutti-frutti (bubblegum flavor), strawberry and banana).

Particles of the type useful in this disclosure comprise a core and a coating essentially completely surrounding the core. The respective flavors may be incorporated therein, either by dissolving the flavor in the material, or by emulsifying it into the material. Typical examples of suitable particles may be found in PCT Publications WO 2005/084458, WO 2006/027542 and WO 2007/274930, the contents whereof are included herein by reference.

In this particular case, a flavor is incorporated into a mixture that corresponds to that of the microparticle core. Around this core is provided a coating, which contains emulsified flavor, which may be of a different kind

The coating is one that will dissolve within a desired time in the mouth, such that the indicator flavor in the core will then be released. The time of the release depends on the nature and thickness of the coating. However, the provision of any suitable release time is within the ordinary skill of the art, with only routine experimentation.

Non-limiting examples of core materials include gelatine, starch grains or grits, and fats with melting points higher than 35° C.

Non-limiting examples of coating materials include carnuba wax, edible plant oils (palm oil, cottonseed oil), miglyol (medium chain triglyceride—MCT), etc.), methyl cellulose or ethyl cellulose.

More than one such coating may be applied, for example, a wax coating may be subsequently coated with a methyl cellulose coating. The nature of the coating will influence the flavor release. For example, flavor in a fat coating will have more delayed, long-lasting release, whereas flavor release in a methyl cellulose coating will be much quicker. Thus, a microparticle having two such coatings and containing the same flavor will provide both immediate and extended flavor release, prior to release of flavor from the core.

The diameter of the microparticles may vary over a wide range, but are typically chosen from the size range of from 50-1500 microns, particularly from 425-850 microns. However, these ranges are largely dictated by processing conditions and not by any limitations, so it is possible in many circumstances to utilise microparticles with sizes outside this range. Nevertheless, the larger the particle, the more detectable it is by tongue, and the result of an excessively large proportion of very large microparticles can be an undesirable “gritty” sensation.

The proportions of the flavors in the microparticles may be chosen so as to provide the necessary strength of flavor. This will depend on the natures of the flavors themselves, and they can be chosen using the ordinary skill of the art. Typical non-limiting flavor levels are 0.1-10% by weight for each flavor.

The dental composition base in which the microparticles are included comprises all the normal ingredients of such compositions in art-recognised proportions. These include a continuous medium (paste or gel) in which all other ingredients are dispersed. These may include surfactants, gelling agents and abrasive agents. Flavors may be included in the continuous medium, in addition to those in the microparticles, but in a particular embodiment, all of the flavor is present in the microparticles.

The disclosure is further described with reference to the following worked examples, which depict particular embodiments, and which are not intended to be in any way limiting.

### EXAMPLE 1

Microparticles were made according to the process described in Example 1 of WO 2005/084458. The ingredients used were:
(a) particle core:
gelatine (43%),
gum Arabic (3%),
xylitol (13%),
palm oil (17%),
mint flavor (18%);
   (b) particle coating:
methyl cellulose (5%),
orange flavor (1%).

All proportions are by weight of the total microparticle composition. In both core and coating, the materials are dissolved or dispersed in a suitable quantity of water. For the core, about twice the weight of the materials of water is used, for the coating from 10-20 times the weight of the materials. Provided the resulting dispersion or solution can be used in the process, the precise amount is not critical.

The microparticles thus prepared had an average diameter of 425-850 microns, with a final flavor loading in the core of 7% (because of processing losses).

### EXAMPLE 2

Example 1 was repeated, with the substitution of an equal proportion of MCT (medium-chain triglyceride)/palm oil for the methyl cellulose of Example 1. The microparticles thus obtained had a similar average diameter to those of Example 1.

### EXAMPLE 3

The microparticles of both Examples 1 and 2 were each incorporated into a toothpaste formulation as follows:

30 To 100 g of a commercially-available toothpaste with a mild bubble gum flavor were added flavor-coated microparticles at a concentration of 1% by weight of the toothpaste. The toothpaste and particles were mixed until an evenly distributed mixture was obtained.

The toothpastes were then brushed by a team of panellists. All panellists reported that the flavor changed noticeably from orange to mint at a time between 100 and 120 seconds from the commencement of brushing, corresponding to a desirable brushing time. Although the embodiments have been described in detail through the above description and the preceding examples, these examples are for the purpose of illustration only and it is understood that variations and modifications can be made by one skilled in the art without departing from the scope of the disclosure. It should be understood that the embodiments described above are not only in the alternative, but can be combined.

1. A dental brushing composition comprising a brushing composition base additionally containing a plurality of microparticles, the microparticles comprising a core, a coating essentially completely surrounding the core, and two different flavors, wherein the two different flavors comprise a general flavor and an indicator flavor, the general flavor being in the coating and the indicator flavor being in the core, the coating being essentially completely soluble under brushing conditions in a desired brushing time.

2. The composition according to claim 1, in which the core is composed of materials selected from gelatine, starch grains or grits, and fats with melting points higher than 35° C.

3. The composition according to claim 1, in which the coating material is selected from materials selected from camuba wax, edible plant oils, medium chain triglyceride, methyl cellulose and ethyl cellulose.

4. The composition according to claim 1, in which more than one coating material is used.

5. The composition according to claim 4, in which there are two coatings, a wax coating followed by a methyl cellulose coating.

6. The composition according to claim 1, in which the microparticles are chosen from the size range of from 50-1500 microns.

7. The composition according to claim 1, in which the proportion of each flavour in the microparticles is from 0.1-10% by weight.

8. The composition according to claim 1, in which the brushing composition base contains flavour, in addition to the flavours contained in the microparticles.

9. The composition according to claim 1, in which all of the flavor in the composition is present in the microparticles.

10. A method of indicating the attainment of a desired brushing time with a brushing dental composition, comprising the release into a mouth of two different encapsulated flavors, wherein the two different flavors comprise a general flavor and an indicator flavor, the indicator flavor indicating the attainment of the brushing time, wherein both flavors are incorporated in the same microparticles, the microparticles comprising a core and a coating essentially completely surrounding the core, the core comprising the indicator flavor and the coating comprising the general flavor, the coating being essentially completely soluble under brushing conditions in a desired brushing time.

11. A method comprising providing a brushing dental composition capable of indicating the attainment of a desired brushing time therewith, said indicating comprising releasing into a mouth two different encapsulated flavors, wherein the two different flavors comprise a general flavor and an indicator flavor, the indicator flavor indicating the attainment of the brushing time, wherein both flavors are incorporated in a single population of microparticles, the microparticles comprising a core and a coating essentially completely surrounding the core, the core comprising the indicator flavor and the coating comprising the general flavor, the coating being essentially completely soluble under brushing conditions in the desired brushing time.

12. The composition according to claim 1, in which the microparticles are chosen from the size range of from 425-850 microns.
